SEQUENCE LISTING (2 sequences)

SEQ ID NO: 1 - protein, 857 aa, Escherichia coli
MRLDRLTNKFQLALADAQSLALGHDNQFIEPLHLMSALLNQEGGSVSPLLTSAGINAGQLRTDINQALNRLPQVEGTGGDVQPSQDLVRVLNLCDKLAQKRGDNFISSELFVLAALESRGTLADILKAAGATTANITQAIEQMRGGESVNDQGAEDQRQALKKYTIDLTERAEQGKLDPVIGRDEEIRRTIQVLQRRTKNNPVLIGEPGVGKTAIVEGLAQRIINGEVPEGLKGRRVLALDMGALVAGAKYRGEFEERLKGVLNDLAKQEGNVILFIDELHTMVGAGKADGAMDAGNMLKPALARGELHCVGATTLDEYRQYIEKDAALERRFQKVFVAEPSVEDTIAILRGLKERYELHHHVQITDPAIVAAATLSHRYIADRQLPDKAIDLIDEAASSIRMQIDSKPEELDRLDRRIIQLKLEQQALMKESDEASKKRLDMLNEELSDKERQYSELEEEWKAEKASLSGTQTIKAELEQAKIAIEQARRVGDLARMSELQYGKIPELEKQLEAATQLEGKTMRLLRNKVTDAEIAEVLARWTGIPVSRMMESEREKLLRMEQELHHRVIGQNEAVDAVSNAIRRSRAGLADPNRPIGSFLFLGPTGVGKTELCKALANFMFDSDEAMVRIDMSEFMEKHSVSRLVGAPPGYVGYEEGGYLTEAVRRRPYSVILLDEVEKAHPDVFNILLQVLDDGRLTDGQGRTVDFRNTVVIMTSNLGSDLIQERFGELDYAHMKELVLGVVSHNFRPEFINRIDEVVVFHPLGEQHIASIAQIQLKRLYKRLEERGYEIHISDEALKLLSENGYDPVYGARPLKRAIQQQIENPLAQQILSGELVPGKVIRLEVNEDRIVAVQ

SEQ ID NO: 2 - protein, 13 aa, Homo sapiens
SYSMEHFRWGKPV

(19) United States
(12) Patent Application Publication
(10) Pub. No.: US 2021/0401020 A1
(43) Pub. Date: Dec. 30, 2021

(54) HAFNIA ALVEI FORMULATIONS

(71) Applicants: TARGEDYS, Rouen (FR); CENTRE HOSPITALIER UNIVERSITAIRE DE ROUEN, Rouen (FR); INSERM (INSTITUT NATIONAL DE LA SANTÉ ET DE LA RECHERCHE MÉDICALE), Paris Cedex 13 (FR); UNIVERSITÉ DE ROUEN, Mont-Saint-Aignan (FR)

(72) Inventors: Clémentine PICOLO, Rouen (FR); Grégory LAMBERT, Châtenay-Malabry (FR); Romain LEGRAND, Saint-Etienne-du-Rouvray (FR); Nicolas LUCAS, Rouen (FR)

(73) Assignees: TARGEDYS, Rouen (FR); CENTRE HOSPITALIER UNIVERSITAIRE DE ROUEN, Rouen (FR); INSERM (INSTITUT NATIONAL DE LA SANTÉ ET DE LA RECHERCHE MÉDICALE), Paris Cedex 13 (FR); UNIVERSITÉ DE ROUEN, Mont-Saint-Aignan (FR)

(21) Appl. No.: 17/290,385

(22) PCT Filed: Nov. 28, 2019
(86) PCT No.: PCT/EP2019/082949

§ 371 Date: Apr. 30, 2021

(30) Foreign Application Priority Data
Nov. 28, 2018 (EP) 18208983.9

(51) Int. Cl. A23L 33/135; A23L 33/00; A61K 35/741; A61K 9/48

(52) U.S. Cl. CPC A23L 33/135; A23L 33/30; A61K 35/741; A61K 9/4891; A61K 9/485; A61K 9/4866; A61K 9/4858

(57) ABSTRACT

A composition essentially made of a Hafnia alvei probiotic strain expressing the ClpB protein; wherein the ClpB protein is in an amount of at least 0.7% (w/w) in weight relative to the total weight of the composition; and the ratio of the total number of Hafnia alvei Colony Forming Units to the total Hafnia alvei cell number ranges from 10⁻⁴ to 0.8. Also, oral dosage forms, namely gastro-resistant capsules including the composition of essentially made of a Hafnia alvei probiotic strain expressing the ClpB protein.

### FIELD OF INVENTION

The present invention relates to probiotic strain compositions, namely Hafnia alvei compositions and oral formulations thereof.

### BACKGROUND OF INVENTION

The international WO2017/174658 patent application discloses pharmaceutical and food compositions comprising Hafnia alvei for inducing satiation prolonging satiety and improving body-weight composition in subjects in need thereof.

Enterobacteriaceae strains (enterobacteria) such as Hafnia alvei express the chaperone protein ClpB. The international patent application WO2016/193829 discloses that the effects of the ClpB protein on the food-intake is dose-dependent. Furthermore, the international patent application WO2018/185080 shows that ClpB protein fragments are also bioactive.

WO2016/193829 discloses that the effect of the enterobacteria probiotic strain depends on the bacterial growth phase of the enterobacteria. In view of optimizing the effect of Hafnia alvei probiotic compositions, one skilled in the art cannot predict neither the ClpB expression by the probiotic strain nor the concomitant biological effect on the body weight composition. However, suitable compositions and formulations comprising Hafnia alvei is an unmet need.

Prior art disclosures concerning the probiotics expressing weight-loss inducing metabolites teach towards the inactivation of the probiotic strains. Indeed, Akkermansia muciniphila is found to be more effective when administered in an inactivated, non-replicative and non-colony-forming state (Plovier et al. 2017 Nature Medicine, 23(1), pp. 107-113).

It was surprisingly found by the Applicant, that contrary to the teaching of the prior art, the effects of Hafnia alvei on a subject's body-weight composition positively corelate with the number of Colony Forming Units (CFU) of the probiotic composition relative to the total cells of the probiotic composition. In particular, the effects of the Hafnia alvei probiotic compositions were particularly advantageous when the ratio of the total number of Hafnia alvei Colony Forming Units to the total Hafnia alvei cell number was at least 10⁻⁴. As opposed to other probiotic strains, H. alvei live and replicatively-active cells can reach the colon and exert their beneficial metabolic activities.

Without willing to be bound by a theory, the present invention ensures both a direct effect on body weight-management via the ClpB vectorized by H. alvei cells and a long-term effect thanks to the attaining of replicatively and metabolically active cells in the distal parts of the intestine. In other terms, the composition according to the invention ensures the presence of ClpB protein in the proximal intestine and the suitable Hafnia alvei growth phase and further ClpB expression in the subject's distal intestine, that how ensuring the optimal effects on the subject's body weight composition.

A further object of the present invention is the supply of oral dosage forms that shall guarantee the liberation of Hafnia alvei in the intestine where the probiotic effects shall take place as generally recognized in the art. Preliminary studies of the Applicant showed that Hafnia alvei ClpB is sensitive to the acidic conditions of the stomach. The oral dosage form according to the invention maximizes the protection of the probiotic strain and the ClpB protein during the passage through the stomach. Thus, the oral dosage form of the invention ensures the probiotic efficiency of Hafnia alvei.

It was surprisingly found that the invention's oral dosage form not only protects the stability of the probiotic strain but also enhances the stability of the bioactive ClpB fragments.

### SUMMARY

The present invention relates to Hafnia alvei compositions and Hafnia alvei oral formulations. In particular, the present invention is defined by the claims.

The present invention relates to a composition essentially consisting of Hafnia alvei probiotic strain; said strain expressing the ClpB protein; wherein the ClpB protein is in an amount of at least 0.7% (w/w) in weight relative to the total weight of the composition; and the ratio of the total number of Hafnia alvei Colony Forming Units to the total Hafnia alvei cell number ranges from 10⁻⁴ to 0.8.

In one embodiment, the number of Hafnia alvei Colony Forming Units cells is equal or superior to 10⁶ per gram of composition.

In one embodiment, the total number Hafnia alvei cell number is equal or superior to 10¹¹ per gram of composition.

In one embodiment, the Hafnia alvei strain is freeze-dried.

The present invention further relates to a pharmaceutical or nutraceutical composition, comprising from 5 to 30% (w/w) of the composition as defined above, said pharmaceutical or nutraceutical composition further comprising at least one pharmaceutically or nutraceutically acceptable excipient.

In one embodiment, the at least one pharmaceutically or nutraceutically acceptable excipient is selected from at least one anti-adherent and at least one texturizing agent.

In one embodiment, the at least one anti-adherent is magnesium stearate.

In one embodiment, the at least one texturizing agent is a modified starch.

In one embodiment, the pharmaceutical or nutraceutical composition further comprises zinc and/or chrome.

In one embodiment, the zinc and/or chrome are in the form of organic salts.

In one embodiment, the pharmaceutical or nutraceutical comprises:
from about 10% to about 15% (w/w) of a Hafnia alvei composition according to any to the invention;
from about 80 to about 85% (w/w) of modified starch;
about 0.5 to about 1.5% (w/w) of magnesium stearate;
about 2.0 to about 3.0% (w/w) of a zing organic salt selected from zinc bisglycinate; and
from about 0.01 to about 0.03% (w/w) of a chrome organic salt selected from chrome picolinate; in weight relative to the total weight of the composition.

The present invention further relates to an oral dosage form selected from capsules and tables comprising the pharmaceutical or nutraceutical composition as defined above.

In one embodiment, the oral dosage form is capsules.

In one embodiment, the oral dosage form is coated with an enteric coating.

In one embodiment, the enteric coating comprises hydroxypropyl methyl-cellulose and gellan gum.

The present invention further relates to a blister comprising at least one oral dosage form as defined above.

### DEFINITIONS

In the present invention, the following terms have the following meanings:
“About” preceding a figure means plus or less 10% of the value of said figure.
“Food composition”, “dietary supplements”, “nutraceutical composition” and “functional food” are interchangeable and refer to any substance containing nutrients, whether for human or animal consumption, whether comprised of a single ingredient or a mixture of ingredients, whether liquid, liquid containing or solid, whether primarily carbohydrate, fat, protein or any mixture thereof, whether edible per se or requiring processing like cooking, mixing, cooling, mechanical treatment and the like.
“Pharmaceutically” or “nutraceutically acceptable” refer to molecular entities and compositions that do not produce an adverse, allergic or other untoward reaction when administered to a subject, especially a human, as appropriate. A pharmaceutically acceptable carrier or excipient refers to a non-toxic solid, semi-solid or liquid filler, diluent, encapsulating material or formulation auxiliary of any type. Pharmaceutically or nutraceutically acceptable excipients that may be used in the compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, silica, colloidal silica, magnesium trisilicate, polyvinyl pyrrolidone, cellulose-based substances (for example sodium carboxymethylcellulose), modified starches, polyethylene glycol, polyacrylates, waxes, polyethylene- polyoxypropylene-block polymers, polyethylene glycol and wool fat. In the pharmaceutical or nutraceutical compositions of the present invention, the active principle, alone or in combination with another active principle, can be administered in a unit administration form, as a mixture with conventional pharmaceutical or nutraceutical supports, to animals and human beings. Suitable unit administration forms comprise oral-route forms such as tablets, gel capsules, powders, granules and oral suspensions or solutions. The pharmaceutical or nutraceutical compositions may further contain antioxidant agents such as ascorbic acid, ascorbyl palmitate, BHT, potassium sorbate or Rosmarinus officinalis extracts. The pharmaceutical compositions may further contain flavour agents such as sugars, fruit or tea flavourings. Compositions comprising probiotics according to the invention can be prepared in water suitably mixed with a with a gelling agent, preferably modified starch. In one embodiment, the vehicle further comprises hydroxypropylmethylcellulose. In one embodiment, the vehicle does not comprise hydroxypropylmethylcellulose. Prolonged absorption of the injectable compositions can be brought about by the use in the compositions of agents delaying absorption, namely coatings as hereinafter described. The person responsible for administration will, in any event, determine the appropriate dose for the individual subject.

### DETAILED DESCRIPTION

The Applicants have demonstrated that the effects of Hafnia alvei on a subject's body-weight composition depends on the number of Colony Forming Units (CFU) of the probiotic composition relative to the total cells of the probiotic composition. In particular, the effects of the Hafnia alvei probiotic compositions were particularly advantageous when the ratio of the total number of Hafnia alvei Colony Forming Units to the total Hafnia alvei cell number was at least 10⁻⁴.

Accordingly, one aspect of the present invention relates to a composition essentially consisting of Hafnia alvei probiotic strain; said strain expressing the ClpB protein; wherein the ClpB protein is in an amount of at least 0.7% (w/w) in weight relative to the total weight of the composition; and the ratio of the total number of Hafnia alvei Colony Forming Units to the total Hafnia alvei cell number is at least 10⁻⁴.

### Composition

In one embodiment, the composition essentially consists of or comprises at least 75% (w/w) Hafnia alvei probiotic strain. In one embodiment, the composition comprises at least 75%, at least 80% (w/w), at least 85% (w/w), at least 90% (w/w), at least 95%, at least 96%, at least 97%, at least 98% (w/w) or, at least 99% of a probiotic strain, preferably Hafnia alvei probiotic strain.

In one embodiment, the composition is a solid composition.

In one preferred embodiment, the composition is a pulverulent composition (powder).

In one embodiment, the pulverulent composition (powder) presents a particle size distribution wherein particles smaller than 500 μm represent less than 80% of the particle size distribution.

In one particular embodiment, the composition is a freeze-dried composition.

In one embodiment, the composition presents more than 95% (w/w) of dry matter, in weight relative to the total composition.

In one preferred embodiment, the composition presents a water activity value (Aw) not exceeding 0.05, preferably not exceeding 0.03, even more preferably not exceeding 0.02.

### Hafnia alvei

Hafnia alvei is a facultatively anaerobic rod-shaped bacillus belonging to the family of Enterobacteriaceae.

In one embodiment, Hafnia alvei is a food-grade Hafnia alvei strain.

In one embodiment, Hafnia alvei is Hafnia alvei 4597 strain.

In one embodiment, Hafnia alvei is not sterilized or pasteurized. In the context of the present invention, pasteurization is a treatment intended to inactivate the bacterial metabolic and/or replicative capacities which commonly consists in heating at a temperature from 50° C. to 100° C. for at least 10 minutes. Pasteurization effects reflect to the reduced or inexistent number of CFUs after such treatment. Sterilization such as autoclaving is a treatment intended to destroy, kill or inactivate all life forms and other biological agents, usually by heating at a temperature from more than 100° C., preferably more than or equal to 121° C. for at least 15 minutes under pressurized conditions. Both pasteurization and sterilization will not only alter the viability of the bacteria but also degrade and partially inactivate the ClpB protein itself.

### ClpB

WO2017/174658 describes that Hafnia alvei is a ClpB-protein-expressing probiotic strain.

As used herein, the term “ClpB” has its general meaning in the art and is also known as heat shock protein F84.1 which is a member of the Hsp100/ClpB family of hexameric AAA+−ATPases. ClpB has been described as an essential factor for acquired thermotolerance several Gram-negative and Gram-positive bacteria. Typically, the amino acid sequence of chaperone protein ClpB comprises or consists of an amino acid sequence 96 to 100% identical to the amino acid sequence of SEQ ID NO: 1. Preferably, the amino acid sequence of ClpB is 96, 97, 98, 99 or 100% identical to the amino acid sequence 540-550 (ARWTGIPVSR) of SEQ ID NO: 1.

In one embodiment, the ClpB protein designates the 96 kDa peptide of SEQ ID NO: 1.

In the context of the present application, the percentage of identity is calculated using a global alignment (i.e. the two sequences are compared over their entire length). Methods for comparing the identity of two or more sequences are well known in the art. The «needle» program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is, for example, available on the ebi.ac.uk world wide web site. The percentage of identity in accordance with the invention is preferably calculated using the EMBOSS: needle (global) program with a “Gap Open” parameter equal to 10.0, a “Gap Extend” parameter equal to 0.5, and a Blosum62 matrix.

According to the invention the ClpB protein mimic the alpha-MSH protein for inducing satiation. Thus, in some embodiments, the ClpB protein of the present invention is recognized by an anti-alpha-MSH antibody.

In one embodiment, the ClpB protein designates the 96 kDa peptide of SEQ ID NO: 1.

In one embodiment, the ClpB protein designates the ClpB fragments of 70, 60, 45, 40, 37, 35, 25 and 17 kDa fragments. Such fragments are recognized by an anti-alpha-MSH antibody. In one embodiment, the ClpB fragments are selected from the fragments of 70, 40, 37 and 25 kDa fragments.

In one embodiment, the ClpB protein designates alpha-MSH antibody cross-reacting dimers or precursors of ClpB and fragments thereof. In one embodiment, such dimers or precursors are selected from the fragments of 100, 125, 130 and 150 kDa.

Typically, the antibody is a monoclonal antibody. In some embodiments, the antibody is a polyclonal antibody such as polyclonal rabbit anti-α-MSH IgG (1:1000, Peninsula Laboratories, San Carlos, Calif., USA). The amino acid sequence of α-MSH preferably comprises or consists of the amino acid sequence SYSMEHFRWGKPV (SEQ ID NO: 2) (Gen Pept Sequence ID, PRF: 223274, as available on Dec. 2, 2013).

SEQ ID NO: 1:
MRLDRLTNKF QLALADAQSL ALGHDNQFIE PLHLMSALLN
QEGGSVSPLL TSAGINAGQL RTDINQALNR LPQVEGTGGD
VQPSQDLVRV LNLCDKLAQK RGDNFISSEL FVLAALESRG
TLADILKAAG ATTANITQAI EQMRGGESVN DQGAEDQRQA
LKKYTIDLTE RAEQGKLDPV IGRDEEIRRT IQVLQRRTKN
NPVLIGEPGV GKTAIVEGLA QRIINGEVPE GLKGRRVLAL
DMGALVAGAK YRGEFEERLK GVLNDLAKQE GNVILFIDEL
HTMVGAGKAD GAMDAGNMLK PALARGELHC VGATTLDEYR
QYIEKDAALE RRFQKVFVAE PSVEDTIAIL RGLKERYELH
HHVQITDPAI VAAATLSHRY IADRQLPDKA IDLIDEAASS
IRMQIDSKPE ELDRLDRRII QLKLEQQALM KESDEASKKR
LDMLNEELSD KERQYSELEE EWKAEKASLS GTQTIKAELE
QAKIAIEQAR RVGDLARMSE LQYGKIPELE KQLEAATQLE
GKTMRLLRNK VTDAEIAEVL ARWTGIPVSR MMESEREKLL
RMEQELHHRV IGQNEAVDAV SNAIRRSRAG LADPNRPIGS
FLFLGPTGVG KTELCKALAN FMFDSDEAMV RIDMSEFMEK
HSVSRLVGAP PGYVGYEEGG YLTEAVRRRP YSVILLDEVE
KAHPDVFNIL LQVLDDGRLT DGQGRTVDFR NTVVIMTSNL
GSDLIQERFG ELDYAHMKEL VLGVVSHNFR PEFINRIDEV
VVFHPLGEQH IASIAQIQLK RLYKRLEERG YEIHISDEAL
KLLSENGYDP VYGARPLKRA IQQQIENPLA QQILSGELVP
GKVIRLEVNE DRIVAVQ

As used herein, “amino acids” are represented by their full name, their three letter code or their one letter code as well known in the art Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

As used herein, the term “amino acids” includes both natural and synthetic amino acids, and both D and L amino acids. “Standard amino acid” or “naturally occurring amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. For example, naphtlylalanine can be substituted for tryptophan to facilitate synthesis. Other synthetic amino acids that can be substituted include, but are not limited to, L-hydroxypropyl, L-3,4-dihydroxyphenylalanyl, alpha-amino acids such as L-alpha-hydroxylysyl and D-alpha-methylalanyl, L-alpha-methylalanyl, beta-amino acids, and isoquinolyl.

As used herein, “amino acid” also encompasses chemically modified amino acids, including but not limited to salts, amino acid derivatives (such as amides), and substitutions. Amino acids contained within the polypeptides of the present invention, and particularly at the carboxy- or amino-terminus, can be modified by methylation, amidation, acetylation or substitution with other chemical groups which can change the polypeptide's circulating half-life without adversely affecting their activity. Additionally, a disulfide linkage may be present or absent in the polypeptides of the invention.

### ClpB Amount

The present invention points out a composition of Hafnia alvei that comprise ClpB protein is in an amount of at least 0.7% (w/w) in weight relative to the total weight of the composition. Typically, the ClpB protein is in an amount equal or superior to 0.7% (w/w), preferably equal or superior to 0.8% (w/w), even more preferably equal or superior to 0.9% (w/w) in weight relative to the total weight of the composition.

In one embodiment, the ClpB protein is in an amount ranging:
from 0.7% to 2.0 (w/w);
from 0.8% to 2.0 (w/w);
from 0.8% to 1.8 (w/w);
from 0.8% to 1.5 (w/w);
from 0.9% to 2.0 (w/w);
from 0.9% to 1.8 (w/w); or
from 0.9% to 1.5 (w/w);
   in weight relative to the total weight of the composition.

It was surprisingly found by the applicant, that the biological effects of Hafnia alvei are CFU (Colony Forming Units)-dependent and total number Hafnia alvei cell number-dependent.

Thus, the composition of the invention is further characterized by the number of Hafnia alvei Colony Forming Units as well as the total number Hafnia alvei cell number.

### Ratio

In one preferred embodiment, the ratio of the total number of Hafnia alvei Colony Forming Units (CFU) to the total Hafnia alvei cell number is at least 10⁻⁴. In one embodiment, the ratio is at least 2.2 10⁻⁴, preferably at least 2.5 10⁻⁴, at least 3 10⁻⁴ or at least 5 10⁻³. In one embodiment, the CFU to the total Hafnia alvei cell number is at least 5 10⁻⁴.

In one embodiment, the CFU to the total Hafnia alvei cell number ranges from 10⁻⁴ to 1.

In one embodiment, the CFU to the total Hafnia alvei cell number ranges from 5 10⁻⁴ to 1.

In one embodiment, the CFU to the total Hafnia alvei cell number ranges from 10⁻⁴ to 0.5.

In one embodiment, the CFU to the total Hafnia alvei cell number ranges from 5 10⁻⁴ to 0.5.

In one preferred embodiment, the CFU to the total Hafnia alvei cell number ranges from 10⁻⁴ to 0.8.

Without willing to be bound by a theory, the ratio according to the present invention guarantees the optimal ClpB secretion by Hafnia alvei within the intestinal tract of the subject that consumed the composition according to the invention. Thus, Hafnia alvei strains may have a dual role. Firstly, acting as a protective vehicle for the ClpB that was expressed by the strain prior to its administration to the subject. Secondly, the Hafnia alvei forming part of the subject's microbiota, shall continue secreting ClpB under the suitable conditions (stationary phase of the strain's growth phase). It appears that the Hafnia alvei Colony Forming Units to the total Hafnia alvei cell number optimizes said dual role of Hafnia alvei and concomitantly the desired beneficial effects on body weight control.

### CFU

In one embodiment, the number of Hafnia alvei Colony Forming Units cells is equal or superior to 10⁶ per gram of composition. In one embodiment, the number of Hafnia alvei Colony Forming Units cells is equal or superior to 5 10⁶ per gram of composition. In one embodiment, the number of Hafnia alvei Colony Forming Units cells is equal or superior to 10⁷ per gram of composition. In one embodiment, the number of Hafnia alvei Colony Forming Units cells is equal or superior to 5 10⁷ per gram of composition. In one embodiment, the number of Hafnia alvei Colony Forming Units cells is equal or superior to 10⁸ per gram of composition. In one embodiment, the number of Hafnia alvei Colony Forming Units cells is equal or superior to 5 10⁸ per gram of composition. In one embodiment, the number of Hafnia alvei Colony Forming Units cells is equal or superior to 10⁹ per gram of composition. In one embodiment, the number of Hafnia alvei Colony Forming Units cells is equal or superior to 10¹⁰ per gram of composition. In one embodiment, the number of Hafnia alvei Colony Forming Units cells is equal or superior to 10¹¹ per gram of composition

In one embodiment, the number of Hafnia alvei Colony Forming Units cells ranges from about 10⁶ to about 5 10¹¹ about per gram of composition.

In one embodiment, the number of Hafnia alvei Colony Forming Units cells ranges from about 10⁷ to about 5 10¹¹ about per gram of composition.

In one embodiment, the number of Hafnia alvei Colony Forming Units cells ranges from about 10⁷ to about 10¹¹ about per gram of composition.

In one embodiment, the number of Hafnia alvei Colony Forming Units cells ranges from about 10⁶ to about 10⁹ about per gram of composition.

In one embodiment, the number of Hafnia alvei Colony Forming Units cells ranges from about 10⁷ to about 5 10¹¹ about per gram of composition.

In one embodiment, the number of Hafnia alvei Colony Forming Units cells ranges from about 10⁷ to about 10¹¹ about per gram of composition.

CFU count techniques are generally known in the art. In one embodiment, the number of CFU is calculated by counting colonies on petri dishes.

In one particular embodiment, the composition essentially consisting of Hafnia alvei probiotic strain as previously described, wherein:
the ClpB protein is in an amount of at least 0.7% (w/w) in weight relative to the total weight of the composition;
the ratio of the total number of Hafnia alvei Colony Forming Units to the total Hafnia alvei cell number is at least 10⁻⁴; and
the number of Hafnia alvei Colony Forming Units cells is equal or superior to 10⁶ per gram of composition.

In one particular embodiment, the composition essentially consisting of Hafnia alvei probiotic strain as previously described, wherein:
the ClpB protein is in an amount of at least 0.7% (w/w) in weight relative to the total weight of the composition;
the ratio of the total number of Hafnia alvei Colony Forming Units to the total Hafnia alvei cell number ranges from 10⁻⁴ to 0.8 and
the number of Hafnia alvei Colony Forming Units cells is equal or superior to 10⁶ per gram of composition.

In one further particular embodiment, the composition essentially consisting of Hafnia alvei probiotic strain as previously described, wherein:
the ClpB protein is in an amount of at least 0.7% (w/w) in weight relative to the total weight of the composition;
the ratio of the total number of Hafnia alvei Colony Forming Units to the total Hafnia alvei cell number ranges from 5 10⁻⁴ to 0.5 and
the number of Hafnia alvei Colony Forming Units cells is equal or superior to 10⁶ per gram of composition.

### Total Cell Number

One skilled in the art can calculate the total number Hafnia alvei cell number based on the CFU number and the ratio of CFU to the total number Hafnia alvei cell number, as previously described.

In one embodiment, the total number Hafnia alvei cell number is at least 10⁸ per gram of composition.

In one embodiment, the total number Hafnia alvei cell number is at least 10⁹ per gram of composition.

In one preferred embodiment, the total number Hafnia alvei cell number is at least 10¹⁰ per gram of composition.

In one embodiment, the total number Hafnia alvei cell number is at least 5 10¹⁰ per gram of composition.

In one embodiment, the total number Hafnia alvei cell number is equal or superior to 10¹¹ per gram of composition.

In one embodiment, the total number Hafnia alvei cell number ranges from 10⁸ to 10¹¹ per gram of composition.

In one embodiment, the total number Hafnia alvei cell number ranges from 10⁹ to 10¹¹ per gram of composition.

In one embodiment, the total number Hafnia alvei cell number ranges from 10¹⁰ to 10¹¹ per gram of composition.

In one embodiment, the total number Hafnia alvei cell number is about 10⁸, about 10⁹, about 10¹⁰, about 10¹¹ or about 10¹², per gram of composition.

In one embodiment, the total number Hafnia alvei cells comprises alive Hafnia alvei cells, alive but inactive Hafnia alvei cells, disrupted Hafnia alvei cells, dead Hafnia alvei cells and mixtures thereof.

In one embodiment, the total number Hafnia alvei cells is measured by Flow Cytometry. According to such embodiment the total number Hafnia alvei cells comprise, intact Hafnia alvei cells, disrupted Hafnia alvei cells, dead Hafnia alvei cells and mixtures thereof.

In one embodiment, the total number Hafnia alvei cells comprises at least 45% of intact Hafnia alvei cells relative to the total cell population. In one embodiment, the total number Hafnia alvei cells comprises at least 50% of intact Hafnia alvei cells relative to the total cell population. In one embodiment, the total number Hafnia alvei cells comprises at least 65% of intact Hafnia alvei cells relative to the total cell population.

In one embodiment, the total number Hafnia alvei cells comprises at least 45% of intact Hafnia alvei cells and less than 5% of dead Hafnia alvei cells, relative to the total cell population.

In one embodiment, the total number Hafnia alvei cells comprises at least 50% of intact Hafnia alvei cells and less than 5% of dead Hafnia alvei cells, relative to the total cell population.

In one embodiment, the total number Hafnia alvei cells comprises at least 65% of intact Hafnia alvei cells and less than 5% of dead Hafnia alvei cells, relative to the total cell population.

In one embodiment, the total number Hafnia alvei cells comprises at least 45% of intact Hafnia alvei cells and less than 3% of dead Hafnia alvei cells, relative to the total cell population.

In one embodiment, the total number Hafnia alvei cells comprises at least 50% of intact Hafnia alvei cells and less than 3% of dead Hafnia alvei cells, relative to the total cell population.

In one embodiment, the total number Hafnia alvei cells comprises at least 65% of intact Hafnia alvei cells and less than 3% of dead Hafnia alvei cells, relative to the total cell population.

### Food Composition

The present invention further relates to a pharmaceutical or nutraceutical composition comprising the composition of the invention as hereinbefore described.

In one embodiment, pharmaceutical or nutraceutical composition comprises at least 5% (w/w) of the previously described composition, in weight relative to the total pharmaceutical or nutraceutical composition.

In one embodiment, pharmaceutical or nutraceutical composition comprises at least 8% (w/w) of the previously described composition, in weight relative to the total pharmaceutical or nutraceutical composition.

In one embodiment, pharmaceutical or nutraceutical composition comprises at least 10% (w/w) of the previously described composition, in weight relative to the total pharmaceutical or nutraceutical composition.

In one embodiment, pharmaceutical or nutraceutical composition comprises from 5% to 30%(w/w) of the previously described composition, in weight relative to the total pharmaceutical or nutraceutical composition.

In one embodiment, pharmaceutical or nutraceutical composition comprises from 8% to 20%(w/w) of the previously described composition, in weight relative to the total pharmaceutical or nutraceutical composition.

In one embodiment, pharmaceutical or nutraceutical composition comprises from 10% to 15%(w/w) of the previously described composition, in weight relative to the total pharmaceutical or nutraceutical composition.

In one embodiment, pharmaceutical or nutraceutical composition comprises about 9%, about 10%, about 11%, about 12%, about 13%, about 14%, or about 15%(w/w) of the previously described composition, in weight relative to the total pharmaceutical or nutraceutical composition. In one embodiment, pharmaceutical or nutraceutical composition comprises about 10%, about 11% or about 12%, (w/w) of the previously described composition, in weight relative to the total pharmaceutical or nutraceutical composition.

### Excipient

In one embodiment, the pharmaceutical or nutraceutical composition of the invention further comprises at least one pharmaceutically or nutraceutically acceptable excipient.

The pharmaceutical or nutraceutical composition that comprises the bacterial strain, in particular the probiotic bacterial strain, of the present invention typically comprises carriers or vehicles. “Carriers” or “vehicles” mean materials suitable for administration and include any such material known in the art such as, for example, any liquid, gel, solvent, liquid diluent, solubilizer, or the like, which is non-toxic and which does not interact with any components, in particular with the bacterial strain, of the composition in a deleterious manner. Examples of pharmaceutically or nutraceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, hydroxypropylmethyl-cellulose polyvinylpyrrolidone, and the like.

Preliminary results showed that Hafnia alvei strain viability is reduced in the acidic conditions of the stomach.

Thus, the pharmaceutical or nutraceutical composition may further comprise a texturizing agent, preferably a gelling agent, even more preferably a modified starch to protect the probiotic strain from the gastric acid degradation.

In one embodiment, the at least one pharmaceutically or nutraceutically acceptable excipient is a vehicle selected from modified starches. In one embodiment, the vehicle is a pre-gelatinized starch. In one embodiment, the vehicle is a modified maize starch. In one embodiment, the vehicle is a pre-gelatinized maize starch, such as for example Pregeflo®.

In one embodiment, the at least one pharmaceutically or nutraceutically acceptable excipient is not a gelling agent comprising hydroxypropylmethylcellulose.

In one embodiment, the vehicle is in an amount ranging from 70% to 90% (w/w), in weight relative to the total pharmaceutical or nutraceutical composition.

In one embodiment, the vehicle is pre-gelatinized starch in an amount ranging from 70% to 88% (w/w), in weight relative to the total pharmaceutical or nutraceutical composition.

In one embodiment, the vehicle is pre-gelatinized starch in an amount ranging from 80% to 88% (w/w), in weight relative to the total pharmaceutical or nutraceutical composition.

In one embodiment, the vehicle is pre-gelatinized starch in an amount of about 81%, about 82%, about 83%, about 84%, about 85%, about 86%, about 87%, or about 88% (w/w), in weight relative to the total pharmaceutical or nutraceutical composition.

The pharmaceutical or nutraceutical composition may further comprise an anti-adherent agent in order to improve the rheological properties of the pharmaceutical or nutraceutical composition.

In one embodiment, the pharmaceutical or nutraceutical composition comprises at least 0.5 (w/w) of an anti-adherent agent, in weight relative to the total pharmaceutical or nutraceutical composition.

In one embodiment, the anti-adherent agent is magnesium stearate.

In one embodiment, the pharmaceutical or nutraceutical composition comprises about 0.5%, about 0.7%, about 0.8%, about 1.0%, about 1.2% or about 1.5%, (w/w) of an anti-adherent agent, preferably magnesium stearate. In one embodiment, the pharmaceutical or nutraceutical composition comprises about 1.0% (w/w) of magnesium stearate, in weight relative to the total pharmaceutical or nutraceutical composition.

In one embodiment, the pharmaceutical or nutraceutical composition further comprises minerals and micronutrients such as trace elements and vitamins in accordance with the recommendations of Government bodies such as the USRDA. For example, the composition may contain per daily dose one or more of the following micronutrients zinc, chrome, calcium, magnesium, phosphorus, iron, copper, iodine selenium, beta carotene, Vitamin C, Vitamin B1, Vitamin B6 Vitamin B2, niacin, Vitamin B12, folic acid, biotin, Vitamin D or Vitamin E.

In one preferred embodiment, the pharmaceutical or nutraceutical composition further comprises zinc and/or chrome.

In an even more preferred embodiment, the pharmaceutical or nutraceutical composition further comprises organic salts of zinc and/or chrome.

In one preferred embodiment, the pharmaceutical or nutraceutical composition further comprises zinc bisglycinate and/or chrome picolinate.

In one preferred embodiment, the pharmaceutical or nutraceutical composition further comprises zinc bisglycinate and chrome picolinate.

In one preferred embodiment, the pharmaceutical or nutraceutical composition further comprises zinc bisglycinate in an amount ranging from 2 to 4% (w/w) and chrome picolinate in an amount ranging from 0.01 to 0.04% (w/w), in weight relative to the total pharmaceutical or nutraceutical composition.

In one preferred embodiment, the pharmaceutical or nutraceutical composition further comprises zinc bisglycinate in an amount of about 3% (w/w) and chrome picolinate in an amount of about 0.02% (w/w), in weight relative to the total pharmaceutical or nutraceutical composition.

In one preferred embodiment, the pharmaceutical or nutraceutical composition further comprises zinc bisglycinate in an amount of 2.8% (w/w) and chrome picolinate in an amount of about 0.02% (w/w), in weight relative to the total pharmaceutical or nutraceutical composition.

In one embodiment, the pharmaceutical or nutraceutical composition further comprises at least one prebiotic. “Prebiotic” means food substances intended to promote the growth of the probiotic bacterial strain of the present invention in the intestines. The prebiotic may be selected from the group consisting of oligosaccharides and optionally contains fructose, galactose, mannose, soy and/or inulin; and/or dietary fibers.

In one embodiment, the pharmaceutical or nutraceutical composition comprises:
from about 10% to about 15% (w/w) of a Hafnia alvei probiotic strain composition of the invention;
from about 80 to about 85% (w/w) of modified starch;
about 0.5 to about 1.5% (w/w) of magnesium stearate;
about 2.0 to about 3.0% (w/w) of a zing organic salt selected from zinc bisglycinate; and
from about 0.01 to about 0.03% (w/w) of a chrome organic salt selected from chrome picolinate;
   in weight relative to the total weight of the composition.

With the proviso that the total in weight percentage concentrations do not exceed 100%. One skilled in the art can adapt the concentration of each ingredient with in the disclosed ranges so as not to exceed 100%.

In one embodiment, the pharmaceutical or nutraceutical composition comprises:
about 11% (w/w) of a Hafnia alvei probiotic strain composition of the invention;
about 85% (w/w) of modified starch;
about 1% (w/w) of magnesium stearate;
about 2.8% (w/w) of a zing organic salt selected from zinc bisglycinate; and
about 0.02% (w/w) of a chrome organic salt selected from chrome picolinate;
   in weight relative to the total weight of the composition.

### Oral Dosage Form

In a further aspect, the invention relates to oral dosage forms comprising the pharmaceutical or nutraceutical composition as previously described.

In one embodiment, the oral dosage form is selected from tablets and capsules.

In one embodiment, the oral dosage form is coated with an enteric coating.

In one embodiment, the oral dosage form is selected from enterically-coated tablets and enterically-coated capsules.

Suitable coatings for such dosage forms are generally known in the art. In one embodiment, the enteric-coating is selected from Methyl acrylate-methacrylic acid copolymers, Cellulose acetate phthalate (CAP), Cellulose acetate succinate, Hydroxypropyl methyl cellulose phthalate, Hydroxypropyl methyl cellulose acetate succinate (hypromellose acetate succinate), Polyvinyl acetate phthalate (PVAP), Methyl methacrylate-methacrylic acid copolymers, shellac, cellulose acetate trimellitate, Sodium alginate and zein. In one embodiment, the enteric-coating may further comprise a thickening agent selected from starches, pectins and polysaccharides selected from algicinic acid and salts thereof, agar-agar, gelatin, carrageenan, locust vena gum and gellan gum.

The Applicants found out that the enteric coating comprising Hydroxypropyl methyl cellulose and gellan gum is particularly advantageous. Indeed, enteric-coated capsules according to the invention provided an improved stability to the bioactive ClpB and fragments thereof, compared to standard Hydroxypropyl methyl cellulose enteric-coatings.

In one preferred embodiment, the oral dosage form is selected from enterically-coated tablets and enterically-coated capsules, wherein the enteric-coating is a mixture comprising Hydroxypropyl methyl cellulose and gellan gum.

In one preferred embodiment, the oral dosage form is an enterically-coated capsule, wherein the enteric-coating is a mixture comprising Hydroxypropyl methyl cellulose and gellan gum.

In one preferred embodiment, the oral dosage form is an enterically-coated capsule, wherein the enteric-coating is a mixture comprising Hydroxypropyl methyl cellulose and gellan gum.

In one embodiment, the enteric coating is the capsule itself.

In one embodiment, the enteric coating comprises from 85 to 95% Hydroxypropyl methyl cellulose and from 5 to 15% gellan gum (w/w) in weight relative to the enteric-coating or the capsule weight.

In one embodiment, the enteric coating comprises about 95% Hydroxypropyl methyl cellulose and about 5% gellan gum (w/w) in weight relative to the enteric-coating or the capsule weight.

In one embodiment, the enteric-coating is a DRcaps™ capsule commercialized by Capsugel®.

In a last aspect, the invention relates to a blister comprising at least one oral dosage form as previously described.

In one embodiment, the blister comprises at least one capsule as previously described.

In one embodiment, the blister comprises 5, 10, 15, 20, 25, 30, 35, 40, 45, 50, 55, or 60 capsules as previously described.

In one embodiment, the blister comprises 30 capsules as previously described.

One further aspect of the present invention relates to a method of:
reducing fat mass on lean mass ratio;
reducing food intake;
inducing satiation;
stimulating weight loss; or
limiting weight gain.
   in a subject in need thereof comprising administering to the subject an effective amount of the composition, the pharmaceutical or nutraceutical composition or the oral dosage form according to the invention.

In one embodiment, the method is a non-therapeutic method.

### BRIEF DESCRIPTION OF THE DRAWINGS

FIG. 1 is a diagram showing the pH-profile during the experiments under fed conditions with the Simulator of the Human Intestinal Microbial Ecosystem. The pH of the medium was controlled automatically. Arrows indicate the time and corresponding pH of samples taken during the stomach incubation phase (ST0 and ST2) and small intestine incubation phase (SI1, SI2, and SI3).

FIG. 2 is a graph showing the average log (CFU)±stdev (n=3) obtained through spread plating on LB agar (A). Average log (count)±stdev (n=3) of viable bacterial cells (B), non-viable bacterial cells (C) and total bacterial cells (D) obtained through flow cytometry. Data are representative for samples collected from the bacterial powder (Product) and those collected during passage in the stomach (ST0 and ST2) and small intestine (SI1, SI2, and SI3) under fed conditions. Differences in samples (ST0/ST2/SI1/SI2/SI3) as compared to their preceding sample were indicated with *.*: statistically significant change (p<0.05).

FIG. 3 is a graph presenting the difference in log (CFU) obtained through spread plating on LB agar (A) and difference in log (count) of viable bacterial cells (B) over three different time spans, i.e. the stomach, small intestinal, and overall GIT incubation during fed conditions. Statistical differences between ST2-Product, SI3-ST2, and SI3-product were calculated. *: statistically significant change (p<0.05).

FIG. 4 is a graph presenting Average log (CFU)±stdev (n=3) obtained through spread plating on LB agar (A). Average log (count)±stdev (n=3) of viable bacterial cells (B) obtained through flow cytometry. Data are representative for samples collected from the mucin beads after 1 h (SI1), 2 h (SI2), and 3 h (SI3) of small intestinal incubation under fed conditions with mucin beads.

FIG. 5 is a graph showing the High fat diet (HFD) validation. A. Body weight (in g) in mice fed with a high fat/high carbs diet (HFD) (n=67) and in mice fed with a control diet (Ctrl) (n=8).

FIG. 6 is a graph showing the ClpB levels in plasma (above, 6A) and feces (below, 6B) measured after the administration of treatment A and the comparative treatment.

FIG. 7 is a graph showing the relative hormone-sensitive lipase protein (pHSL) expression rate (against actine expression rate as a standard) in obese HFD mice treated with composition A, comparative treatment and control treatment. hormone-sensitive lipase protein and actine expression rates were measured by western blot.

FIG. 8 is a graph presenting the fat mass gain (in g) in high fat diet (HFD)-induced obese mice treated with composition A, comparative treatment and control treatment.

FIG. 9 is a graph showing the efficiency in the inhibition of bodyweight gain of Ob/Ob mice by the G2 treatment (4 10⁹ CFU of Hafnia alvei per gram of the composition) according to the present invention. 2-way ANOVA, p=0.041, Bonferroni post-test, Control vs H. alvei, p<0.05

FIG. 10 is a graph showing the dose-dependent improvement of the lean mass to fat mass ration by the treatment of Ob/Ob mice with compositions of the present invention. Kruskal-Wallis, Dunn's post-test, $$$ p<0.001, $$ p<0.01

FIG. 11 is a graph showing the pixel density of the ClpB protein (96 kDa) and bioactive fragments thereof (70 kDa, 40 kDa, 37 kDa and 25 kDa) past the Gastro-Duodeno-Ileal Model simulation within the oral dosage form according to the invention.

### EXAMPLES

The present invention is further illustrated by the following examples.

### Example 1

### Hafnia alvei Survival and Activity in the Simulator of the Human Intestinal Microbial Ecosystem

This example shows the evaluation of the intestinal fate of a strain of Hafnia alvei during passage through the complete gastrointestinal tract (GIT). First, the viability and functionality of the bacterial strain during passage through the upper GIT under fed conditions, when dosed as a powder formulation, was determined. To do this, under very controlled simulated conditions, the Simulator of the Human Intestinal Microbial Ecosystem (SHIME®) was used. Two sets of upper GIT experiments were performed. During the first set of experiments the passage of H. alvei through the fed upper GIT in the absence of a mucosal layer was tested. During the second set of experiments, a mucosal layer was introduced in the small intestine which allowed to study the capacity of the strain to adhere to the gut wall under relevant physiological conditions. The main end-points were the quantification of culturable bacterial cells (CFU) through spread plating and the quantification of viable and non-viable bacterial cells through live/dead flow cytometry and this both on the luminal samples and mucosal samples. After passage of H. alvei through the upper GIT under fed conditions in the absence of a mucosal layer, the small intestinal suspension was transferred to sterile colonic incubations. This allowed to study the growth and metabolic activity of this bacterial strain under proximal colon simulating conditions. The main end-points were the quantification of culturable bacterial cells through spread plating and the quantification of viable and non-viable bacterial cells through flow cytometry. The metabolic activity of the bacteria strain was assessed by measuring of the pH of the medium and by quantifying the concentrations of short chain fatty acids (SCFA), branched chain fatty acids (BCFA), ammonium, and lactate.

The reactor setup was adapted from the SHIME, representing the gastrointestinal tract (GIT) of the adult human, as described by Molly et al. (Molly, K., M. V. Woestyne, et al. 1993 Applied Microbiology and Biotechnology 39: 254-258.). The SHIME consists of a succession of five reactors simulating the different parts of the human gastrointestinal tract.

The first two reactors are of the fill-and-draw principle to simulate different steps in food uptake and digestion, with peristaltic pumps adding a defined amount of SHIME feed and pancreatic and bile liquid, respectively to the stomach and small intestine compartment and emptying the respective reactors after specified intervals. The last three compartments—continuously stirred reactors with constant volume and pH control—simulate the ascending, transverse and descending colon. Retention time and pH of the different vessels are chosen in order to resemble in vivo conditions in the different parts of the gastrointestinal tract.

### Test Product

The strain of Hafnia alvei was tested to assess its survival and the production of a target protein, while passing through the stomach and small intestine. To each stomach reactor 10¹⁰ CFU of H. alvei, formulated as a powder, was added. The ratio of the of Hafnia alvei CFU to the total Hafnia alvei cell number was of 0.32.

All experiments were performed in biological triplicate to account for biological variability.

### Upper GIT Study

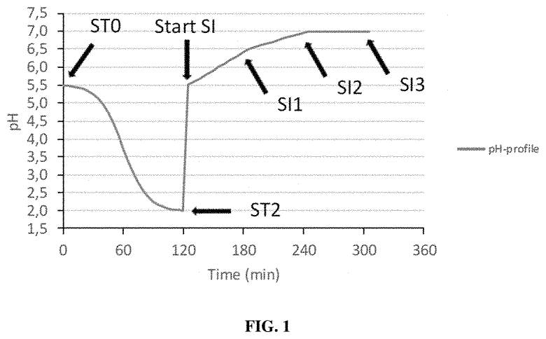

Gastric Phase (Fed State)
Incubation during 2 h at 37° C., while mixing via stirring, with sigmoidal decrease of the pH profile from 5.5 to 2.0 (FIG. 1).
Pepsin is supplied with the activity being standardized by measuring absorbance increase at 280 nm of TCA-soluble products upon digestion of hemoglobin (reference protein).
Addition of phosphatidylcholine.
Addition of the SHIME® nutritional medium containing arabinogalactan, pectin, xylan, starch, glucose, yeast extract, peptone, mucin, and L-cystein-HCl. The salt levels recommended by the consensus method (NaCl and KCl) were implemented.
Sampling: t=0 and 2 h; at these time points the pH of the medium was equal to 5.5±0.05 and 1.99±0.05, respectively.

Small Intestinal Phase (Fed State)
While mixing via stirring, the pH initially automatically increases from 2.0 to 5.5 within a period of 5 minutes after which a gradually increasing pH from 5.5 to 7.0 during an incubation of 3 h at 37° C. is controlled automatically by the software (as shown in FIG. 1).
Regarding pancreatic enzymes both a raw animal pancreatic extract (pancreatin) containing all the relevant enzymes in a specific ratio as well as defined ratios of the different enzymes can be used.
Regarding bile salts, 10 mM bovine bile extract is generally supplemented (bovine bile is a closer match to human than porcine in terms of tauro- and glycocholate)
Addition of mucin coated microcosms to simulate the small intestinal mucus layer (only during one set of upper GIT experiments).
Sampling: t=1 h, 2 h, and 3 h; at these time points the pH of the medium was equal to 6.5±0.05, 7.0±0.05, and 7.0±0.1, respectively.

### CFU Counting

Samples were collected at different stages of the experiment for stomach and small intestine to determine the number of colony forming units of H. alvei by spread plating. The number of colony-forming units of H. alvei contained in the dry powder (product) were also determined. During the experiments with mucus beads, beads were harvested from the reactor. The mucin beads were first washed in a PBS solution. Subsequently, the mucin beads were incubated for 30 min at 37° C. in PBS containing 1% Triton X-100. Ten-fold dilution series were prepared from these samples in phosphate.

### Quantification of Viable and Non-Viable Bacterial Cells by Flow Cytometry

Samples were collected at different stages of the experiment for stomach and small intestine to determine the number of viable and non-viable H. alvei cells by flow cytometry. The number of viable and non-viable H. alvei cells, present in the dry powder (product), were also determined. During the experiments with mucus beads, beads were harvested from the reactor. The mucin beads were first washed in a PBS solution. Subsequently, the mucin beads were incubated for 30 min at 37° C. in PBS containing 1% Triton X-100. A ten-fold dilution series was initially prepared in phosphate buffered saline. Assessment of the viable and non-viable population of the bacteria was done by staining the appropriate dilutions with SYTO 24 and propidium iodide. Samples were analyzed on a BDFacs verse. The samples were run using the high flow rate. Bacterial cells were separated from medium debris and signal noise by applying a threshold level of 200 on the SYTO channel. Optimization of the proper PMT settings and construction of appropriate parent and daughter gates allowed to determine all populations of interest. Results are reported as average log (counts)±stdev of the three independent biological replicates.

Statistically significant differences between the number of CFU and counts of viable and non-viable bacterial cells were determined in between each sampling point and its preceding one during the experiments under fed conditions to demonstrate changes in function of time.

The same differences were determined for the results obtained for the product and the first sampling point of the experiment to demonstrate the immediate influence of the environmental conditions on the survival of the bacterial strain. Furthermore, statistically significant differences were determined for the bacterial strain over three different time spans, i.e. the stomach, small intestinal, and overall gastrointestinal incubation to clearly demonstrate the effect of the residing environmental conditions on the culturability and viability of H. alvei. In terms of statistics, the differences for all data discussed and indicated by “p<0.05” or “*” were significant with a confidence interval of 95%, as demonstrated using a Student's t-test.

### Upper GIT Results

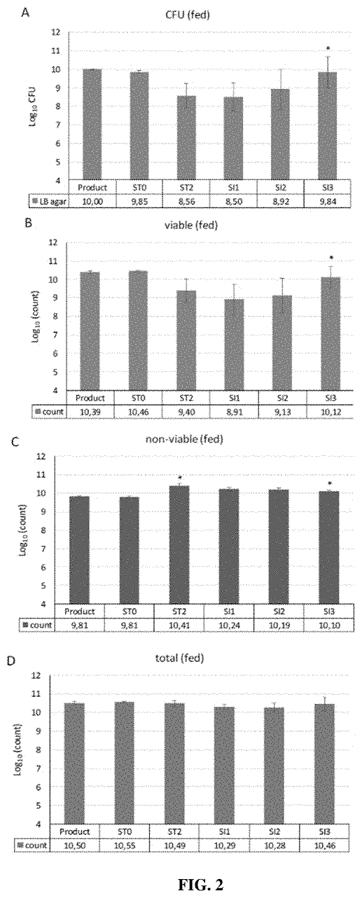

Sampling of the stomach reactor immediately after dosing of the bacterial strain indicated that the number of culturable bacterial cells was equal to the number of culturable bacterial cells present in the product. The same results were obtained for the number of viable, non-viable, and total bacterial cells (FIG. 2). This revealed that there was no immediate effect on the “culturability” and viability of H. alvei once this bacterial strain came into contact with the gastric juice. Under fed conditions the initial pH of the stomach is high (pH value of 5.5) due to the buffering capacity of the ingested food. The continuous secretion of hydrochloric acid in the stomach environment surpasses this initial buffering effect resulting in a sigmoidal decreasing pH of the stomach till a final value of 2.0 over a two-hour period. These low pH values can impose a major stress on bacterial survival in the stomach. Indeed, after 2 h of stomach incubation the number of culturable bacterial cells of H. alvei was decreased and the number of non-viable bacterial cells increased.

After passage through the stomach the bacterial cells enter the small intestinal incubation phase which is marked by a sharp increase in the environmental pH till a value of 5.5. Throughout the small intestinal incubation phase the pH further increases till a final value of 7.0.

Notwithstanding the presence of these beneficial pH conditions in the small intestine, the secretion of bile acids in the small intestinal lumen generally imposes a major challenge on bacterial survival.

Surprisingly, the high concentration of bile acids did not result in a decrease in the number of culturable and viable bacterial cells of H. alvei. Indeed, after 1 h of small intestinal incubation the number of culturable and viable bacterial cells of this strain increased till the end of the stomach incubation. This indicated that H. alvei was not sensitive to bile acids and was even capable to consume the carbohydrate substrates, present in the fed upper GIT, finally resulting in the growth of this bacterial strain.

Throughout the experiment the total number of bacterial cells remained constant indicating that no cell lysis occurred during passage through the fed upper GIT.

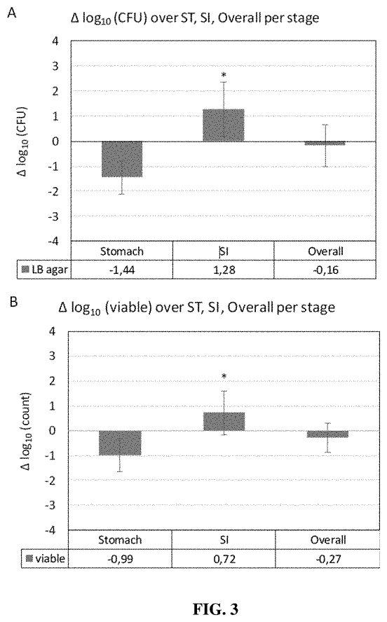

In view of the assessment of the altered levels of the bacterial cells during the stomach (ST2-Product), small intestinal (SI3-ST2) and overall GIT incubation (SI3-Product) are presented in FIG. 3. These results indicated that the number of culturable and viable bacterial cells decreased over the course of the stomach transit. Hence, the strain of H. alvei is sensitive to the low pH conditions of the stomach. During the upper GIT, a net increase in the number of culturable and viable bacterial cells occurred indicating that H. alvei was not sensitive to the high concentrations of bile acids in the small intestine and was capable to grow during the small intestinal transit. As such, the number of culturable and viable bacterial cells of H. alvei was not significantly decreased after a full passage through the upper GIT under fed conditions.

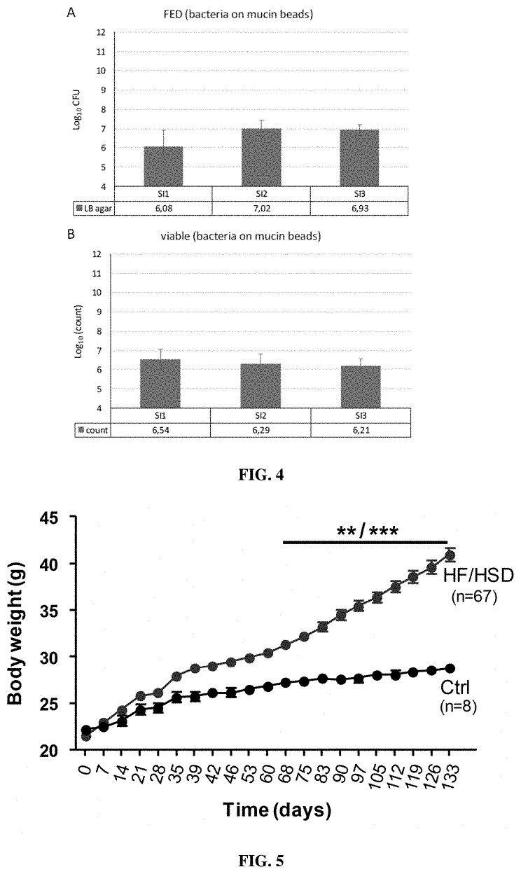

The SHIME assay repeated in the present of mucin beds confirmed the above results. The determination of the number of culturable bacterial cells and viable bacterial cells from samples collected from the initially sterile beads revealed that H. alvei was capable to adhere to the mucin beads and this already after 1 h of small intestinal incubation. Furthermore, this bacterial strain remained attached to the mucin beads after 2 h and 3 h of small intestinal transit (FIG. 4).

### Growth and Metabolic Activity in the Colon

Short-term Colonic Batch Incubations

Short-term colonic batch incubations were performed using a representative colon medium containing host- and diet-derived compounds. To all colonic batch incubations, a centrifuged and autoclaved SHIME suspension was added to provide the bacteria with relevant colonic metabolites. After passage of H. alvei through the upper GIT under fed conditions (in the absence of mucus beads), a part of the small intestinal liquid phase was transferred to colonic reactors containing the colon medium and the sterile SHIME suspension All bottles were incubated for 48 h at 37° C. under anaerobic conditions.

Samples were taken at the start of the incubations (0 h) and after 24 h and 48 h of incubation. The growth of H. alvei under these sterile colonic conditions was determined through quantification of the number of CFU by spread plating and the quantification of the number of viable and non-viable bacterial cells through flow cytometry.

The fermentative activity of H. alvei in the colon was studied by determining the pH of the medium in the colonic reactors. Furthermore, concentrations of acetate, propionate, butyrate, branched chain fatty acids, lactate and ammonium were determined. The experiments were performed in biological triplicate to account for possible biological variability.

Results

During the short-term colonic incubations, H. alvei was capable to grow under proximal colon simulating conditions since during the first 24 h the number of culturable and viable bacterial cells increased (log CFU: T₀: 7.21; T_{24 h}: 8.92 and T_{48 h}: 8.02). In between 24 h and 48 h of colonic incubation the number of culturable and viable bacterial cells decreased. This was mainly due to a conversion of H. alvei from a culturable into a VBNC (viable but non culturable) state since the number of viable bacterial cells decreased less than the number of culturable bacterial cells (log[count of viable cells]: T₀: 7.46; T_{24 h}: 9.06 and T_{48 h}: 8.75). This conversion could be due to the lowering of the pH of the medium (T₀: 6.23; T_{24 h}: 5.96 and T_{48 h}: 5.77) or due to the absence of carbohydrates which were completely consumed during the first 24 h of incubation.

The metabolic activity of H. alvei in the colon was confirmed by the increase of lactate concentrations throughout the colonic incubations (T₀: 0.7 mM; T_{24 h}: 1.77 mM and T_{48 h}: 2.11 mM)

The results of Example 1 show that intact H. alvei cells can vectorize ClpB past the acid conditions of the stomach, since no lysis was observed, and ensure the short-term delivery of ClpB.

Surprisingly, contrary to other bacterial strains that are inactivated by the gastric conditions, H. alvei CFUs attain the proximal intestine where they proliferate and ensure the colonization of the distal parts of the GIT. Hence, the composition according to the invention shall further ensure a more prolonged secretion of ClpB via the CFUs having attained the stationary bacterial growth phase in the colon.

### Example 2

### In Vivo Effect of the Invention's Composition on HFD Mice

This example demonstrates the effect of the Hafnia alvei CFU/total cell ratio on high fat diet-induced obese mice.

One-month-old male C57B16 mice (Janvier Laboratories) were induced with high fat/high carbs diet for 4 weeks. Induction of obesity by high fat diet was validated by measurement of mean body weight (FIG. 5) in a group induced and a group non-induced for obesity.

Mice were then intragastrically gavaged with as follows:

TABLE 1
Treatment groups of the in vivo experiment 2.

| | | | | CFU/total |
|---|---|---|---|---|
| Treatment | Strain | CFU | Total Cells | cells ratio |
| A | H. alvei | 4.0 10⁷ | 8.4 10⁸ | 0.04 |
| Comparative | Inactivated | 0 | 4.8 10⁸ | 0 |
| treatment | H. alvei | | | |
| Control | (MH culture | — | — | — |
| | medium) | | | |

The presence of ClpB in the treatment compositions in treatment A and in the comparative treatment was confirmed by Western-Blot.

Mice were placed individually into the BioDAQ cages (Research Diets) and intragastrically gavaged daily for 6 weeks. At the end of the treatment the mice were euthanized and tissue samples (plasma, colic fecal, epididymal fat) were collected.

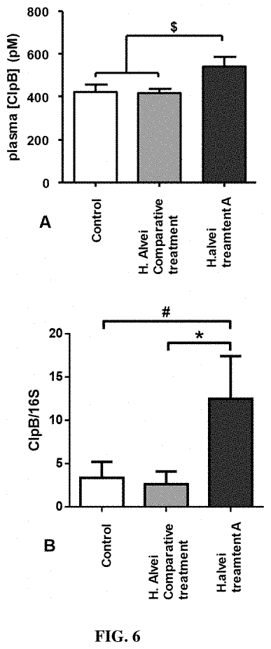

The inventors showed that the comparative treatment did not induce the presence of ClpB in the mice plasma (FIG. 6A) or feces (FIG. 6B), despite the presence of ClpB in such treatment

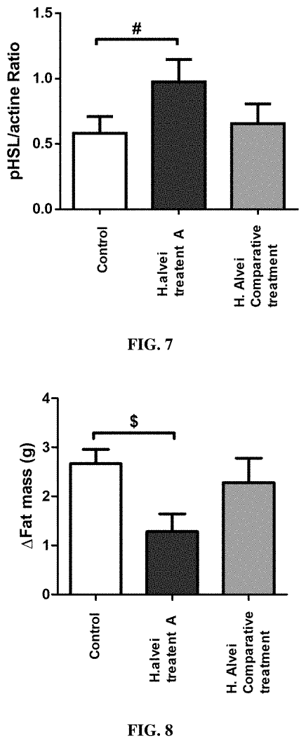

Furthermore, contrary to the composition according to the invention, the comparative composition failed to induce the pHSL expression (FIG. 7) and improve the body composition (fat mass gain inhibition, FIG. 8)

### Example 3

### In Vivo Effect of the Invention's Composition on Ob/Ob Mice

Batch N0717031A of Hafnia alvei composition according to the invention was used to prove the in vivo effects of the composition according to the present invention.

In view of obtaining a negative control, a sample of the batch was inactivated by pasteurization (negative control). The ClpB quantification in the negative control showed a ClpB concentration of 1.56 mg per gram of freeze-dried composition and the CFU/total Hafnia alvei cells ratio was 2.2 10⁻⁴.

Genetically obese ob/ob mice (n=5×15) were acclimated to the animal facility for 1 week. Mice were intragastrically gavaged twice a day for three weeks with the probiotic treatments presented in table 1. At the end of the experiment, mice were euthanized and intestinal and epididymal fat tissue samples were collected.

TABLE 2
Treatment groups of the in vivo experiment 3.

| | | | | | mg |
|---|---|---|---|---|---|
| Treatment | Strain | CFU | Total Cells | CFU | ClpB/g |
| G1 | H. alvei 4597 | 4.0 10¹⁰ | 5.5 10¹⁰ | 0.72 | 9.3 |
| G2 | (Batch 31A) | 4.0 10⁹ | 5.5 10⁹ | | |
| Negative | Inactivated | 8.7 10³ | 4.0 10⁹ | 2.2 10⁻⁷ | 1.56 |
| control 1 | H. alvei 4597 | | | | |
| | (Batch 32A) | | | | |
| Control | (vehicle) | — | — | — | — |

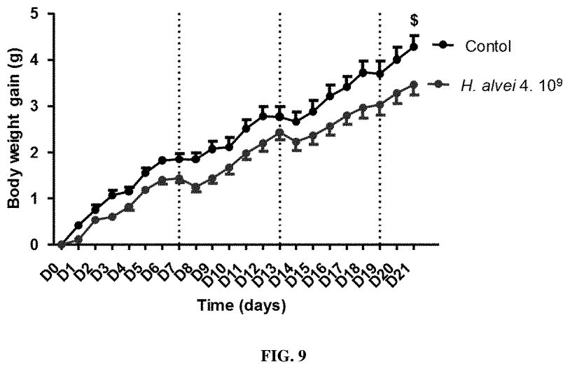

At the end of the treatment the G2 was evaluated for the body weight gain control as a proof of concept. Indeed, Ob/Ob mice presented a significant reduction in weight gain compared to the control group, as presented in FIG. 9.

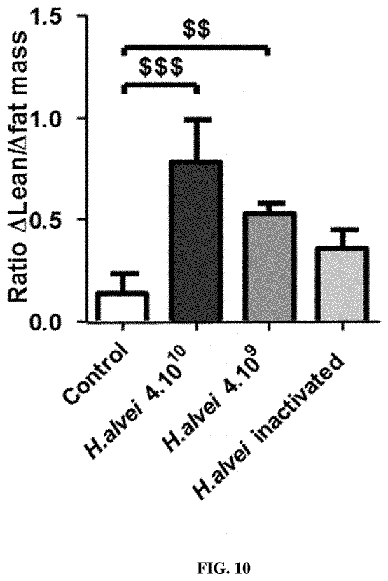

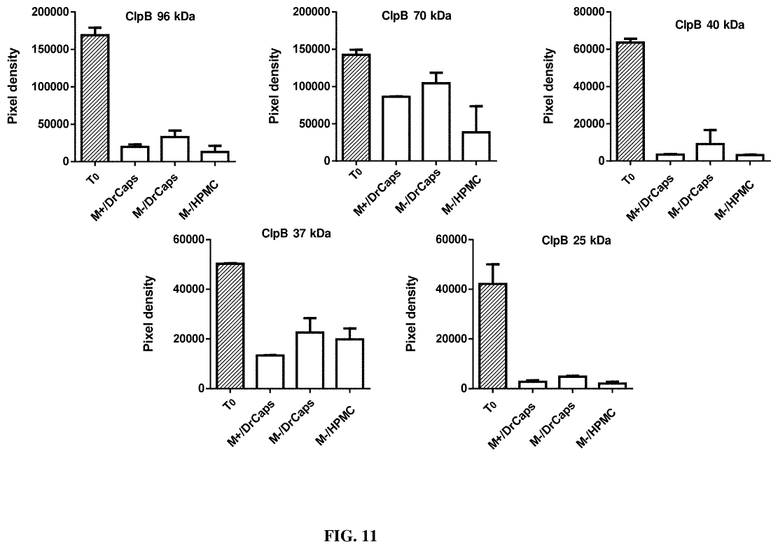

Further analyses were carried out on the body weight analysis. As presented in FIG. 10, the present results confirmed that the treatment with G1-G2 induce a reduction of the body fat mass percentage and the amelioration of the lean mass to fat mass ratio. Interestingly, the improvement in the body composition occurred in a CFU and total cell number dose-dependent manner (G1>G2).

Even more interestingly, the dose dependent effect correlates not only with the ClpB concentration but also with the number of the administrated Hafnia alvei CFU and the ratio of the CFU count over the total Hafnia alvei cells count.

### Example 4

### Batch Production with Improved CFU Count

Given the results of Example 3, different Hafnia alvei bioreactor conditions were tested in view of optimizing the CFU count relative to the total Hafnia alvei cell count.

Preliminary assays showed that the incubation period had no or little effect the CFU count. Furthermore, among the tested conditions, oxygen stress had no beneficial effect on the CFU count. On the contrary, continuous heat stress (38° C.) reduced the CFU count in the bioreactor of laboratory scale. Surprisingly, as presented in table 1 initial 38° C. heat stress or terminal 15° C. stress while the rest of the cell culture maintained at 35° C., presented an improvement of the CFU count.

TABLE 3
Tested bioreactor conditions and CFU count.

| | CFU during Week: | | |
|---|---|---|---|
| Assay | 0 | 2 | 4 |
| Control | 1.16 10¹² | 6.00 10¹¹ | 5.90 10¹¹ |
| A. Terminal 15° C. stress | 1.03 10¹² | 7.05 10¹¹ | 5.70 10¹¹ |
| B. Initial 43° C. stress | 1.32 10¹² | 6.75 10¹¹ | 5.20 10¹¹ |
| C. Initial 38° C. stress | 1.16 10¹² | 6.90 10¹¹ | 6.60 10¹¹ |
| D. Continuous 38° C. stress | 2.70 10¹¹ | 1.10 10¹⁰ | 1.65 10¹⁰ |

TABLE 4
CFU and total cells per gram of obtained compositions under
the tested laboratory scale bioreactor conditions.

| | CFU/g of | Cells/g of | CFU/total |
|---|---|---|---|
| | composition | composition | cell ratio |
| Control | 1.16 10¹² | 1.94 10¹² | 0.60 |
| A. Terminal 15° C. stress | 1.03 10¹² | 1.33 10¹² | 0.77 |
| B. Initial 43° C. stress | 1.32 10¹² | 1.78 10¹² | 0.74 |
| C. Initial 38° C. stress | 1.16 10¹² | 2.06 10¹² | 0.56 |
| D. Continuous 38° C. stress | 2.70 10¹¹ | 4.6 10¹¹ | 0.58 |

Bioreactor conditions A and B were retained since they presented an improved CFU/total cell ratio as presented in table 4.

The quantity of the ClpB protein and fragments thereof was quantified by means of pixel densitometry on the immunoblotted freeze-dried material (Loaded proteins: 50 μg; primary anti-α-MSH primary antibody (polyclonal rabbit; Delphi Genetics)—dilution 1/1000; secondary anti-rabbit-HRP secondary antibody (Dako)—dilution 1/5000; Diluant & blocking buffer TBST-BSA 5%; Exposition time=8 seconds. Films were scanned using ImageScanner III (GE Healthcare) and analyzed for the band pixel density using the ImageQuant TL software 7.0 (GE Healthcare)).

Stress assays A, B improved the CFU/total cell ratio in addition to the ClpB concentration.

Based on those results, two batches were prepared on an industrial scale bioreactor.

CED01 batch characterized by:
about 9 mg of ClpB per gram of lyophilized composition;
5 10¹¹ CFU per gram of lyophilized composition; and
9.7 10¹¹ total Hafnia alvei cells per gram of lyophilized composition.

CED02 batch characterized by:
about 15 mg of ClpB per gram of lyophilized composition;
2.7 10¹¹ CFU per gram of lyophilized composition; and
1.12 10¹² total Hafnia alvei cells per gram of lyophilized composition.

### Example 5

### Oral Dosage Form

Four oral dosage forms we prepared comprising the CED01 batch as hereinbefore described and presented in table 5.

TABLE 5
Screened oral dosage forms.

| | | | Mass % | Mass per |
|---|---|---|---|---|
| Form | Capsule | Constituents | (w/w) | capsule(g) |
| M+/HPMC | HPMC | H. alvei CED01 | 10.6 | 0.05 |
| | Capsule | Pregeflo ® | 81.4 | 0.3852 |
| | | Methocell K100M ® | 7.0 | 0.0331 |
| | | Magnesium stearate | 1.0 | 0.0047 |
| | | Total | 100 | 0.4730 |
| M−/HPMC | HPMC | H. alvei CED01 | 10.6 | 0.05 |
| | Capsule | Pregeflo ® | 88.4 | 0.4183 |
| | | Magnesium stearate | 1.0 | 0.0047 |
| | | Total | 100 | 0.4730 |
| M+/DrCaps ® | DrCaps ® | H. alvei CED01 | 10.6 | 0.05 |
| | | Pregeflo ® | 81.4 | 0.3852 |
| | | Methocell K100M ® | 7.0 | 0.0331 |
| | | Magnesium stearate | 1.0 | 0.0047 |
| | | Total | 100 | 0.4730 |
| M−/DrCaps ® | DrCaps ® | H. alvei CED01 | 10.6 | 0.05 |
| | | Pregeflo ® | 88.4 | 0.4183 |
| | | Magnesium stearate | 1.0 | 0.0047 |
| | | Total | 100 | 0.4730 |

### Example 6

### Gastro-Duodeno-Ileal Model (GDIM)

The oral dosage forms of example 5 were subjected to a GDIM assay. The results of this example allow the selection of the excipients as well as the coating agent or capsule used for an efficient oral administration of the composition according to the present invention.

In brief, humified dosage forms of example were subjected to incubation through three compartments, each simulating the gastric, duodenal and ileal content:
Gastric compartment T₀-T₃₀
44 mL of a solution consisting of NaCl (3 g/L), KCl (1.1 g/L), CaCl₂ (0.15 g/L), pepsin (951 U/mg; 0.003% w/w) Citrate/phosphate buffer 32 mM, pH 3.5. 37° C., rotation at 150 rotations per minute.
Duodenal compartment T₃₀-T₆₀
65 mL of a solution consisting of NaCl (3 g/L), KCl (1.1 g/L), CaCl₂ (0.15 g/L), bile (0.3% w/w), Trypsin (7500 U/mg 0.007% w/w), Di-sodium hydrogen phosphate dihydrate buffer 200 mM. pH 6.5, 37° C., rotation at 150 rotations per minute.
Ileal compartment T₆₀-T₁₂₀
65 mL of a solution consisting of NaCl (3 g/L), KCl (1.1 g/L), CaCl₂ (0.15 g/L), bile (0.3% w/w), Trypsin (7500 U/mg; 0.0056% w/w), Di-sodium hydrogen phosphate dihydrate buffer 200 mM. pH 7.0, 37° C., rotation at 150 rotations per minute.

Past the GDIM assay, samples (n=3) were homogenized in aseptic conditions and the viability of Hafnia alvei past the GDIM was assessed by flow cytometry. The latter analysis showed that all four dosage forms sufficiently protected Hafnia alvei through the gastric pass.

The obtained samples were then lyophilized and the ClpB content was measured in the obtained powders, by densitometry of the Western-blot as previously detailed.

Surprisingly, the formulation of the invention coated with a hydroxypropylmethylcellulose and gellan gum coating (DrCaps®) yielded the best stability of ClpB during the simulated digestion.

More in particular, as shown in FIG. 11, the formulation devoid of texturizing agent (hydroxypropylmethylcellulose, Pregeflo®) showed the a pronounced gastric-resistance not only for the ClpB protein (˜96 kDa) but also for the bioactive ClpB fragments (˜96 kDa, ˜70 kDa, ˜40 kDa, ˜37 kDa and ˜25 kDa).

1-15 (canceled)

16. A composition essentially consisting of Hafnia alvei probiotic strain; said strain expressing the ClpB protein; wherein:
the ClpB protein is in an amount of at least 0.7% (w/w) in weight relative to the total weight of the composition; and
the ratio of the total number of Hafnia alvei Colony Forming Units to the total Hafnia alvei cell number ranges from 10⁻⁴ to 0.8.

17. The composition according to claim 16, wherein the number of Hafnia alvei Colony Forming Units cells is equal or superior to 10⁶ per gram of composition.

18. The composition according to claim 16, wherein the total number Hafnia alvei cell number is equal or superior to 10¹⁰ per gram of composition.

19. The composition according to claim 16, wherein the Hafnia alvei strain is freeze-dried.

20. A pharmaceutical or nutraceutical composition, comprising from 5 to 30% (w/w) of the composition according to claim 16, said pharmaceutical or nutraceutical composition further comprising at least one pharmaceutically or nutraceutically acceptable excipient.

21. The pharmaceutical or nutraceutical composition according to claim 20, wherein said at least one pharmaceutically or nutraceutically acceptable excipient is selected from a group consisting of at least one anti-adherent, at least one texturizing agent, and combinations thereof.

22. The pharmaceutical or nutraceutical composition according to claim 21, wherein said at least one anti-adherent is magnesium stearate.

23. The pharmaceutical or nutraceutical composition according to claim 21, wherein said at least one texturizing agent is a modified starch.

24. The pharmaceutical or nutraceutical composition according to claim 20, further comprising zinc and/or chrome.

25. The pharmaceutical or nutraceutical composition according to claim 24, wherein the zinc and/or chrome are in the form of organic salts. 26 (New). The pharmaceutical or nutraceutical composition according to claim 20, said composition comprising:
from about 10% to about 15% (w/w) of a Hafnia alvei composition essentially consisting of Hafnia alvei probiotic strain; said strain expressing the ClpB protein; wherein: the ClpB protein is in an amount of at least 0.7% (w/w) in weight relative to the total weight of the composition; and wherein the ratio of the total number of Hafnia alvei Colony Forming Units to the total Hafnia alvei cell number ranges from 10⁻⁴ to 0.8;
from about 80 to about 85% (w/w) of modified starch;
from about 0.5 to about 1.5% (w/w) of magnesium stearate;
from about 2.0 to about 3.0% (w/w) of a zing organic salt selected from zinc bisglycinate; and
from about 0.01 to about 0.03% (w/w) of a chrome organic salt selected from chrome picolinate;
in weight relative to the total weight of the composition.

27. An oral dosage form selected from capsules and tables, said dosage form comprising the pharmaceutical or nutraceutical composition according to claim 20.

28. The oral dosage form according to claim 27, said oral dosage form being coated with an enteric coating.

29. The oral dosage form according to claim 27, said oral dosage form being in the form of capsules.

30. The oral dosage form according to claim 27, said enteric coating comprising hydroxypropyl methyl-cellulose and gellan gum.

31. A blister comprising at least one oral dosage form according to claim 27.
